(19) United States
(12) Patent Application Publication
(10) Pub. No.: US 2025/0213099 A1
(43) Pub. Date: Jul. 3, 2025

(54) SYSTEMS AND METHODS FOR ROBOTIC ENDOSCOPE BENDING SECTION

(71) Applicant: Noah Medical Corporation, San Carlos, CA (US)

(72) Inventors: Maziyar Keshtgar, San Bruno, CA (US); Ka Chun Wong, Foster City, CA (US)

(21) Appl. No.: 19/082,739

(22) Filed: Mar. 18, 2025

Related U.S. Application Data
(63) Continuation of application No. PCT/US2023/079054, filed on Nov. 8, 2023.
(60) Provisional application No. 63/383,788, filed on Nov. 15, 2022.

(51) Int. Cl. A61B 1/008; A61B 1/00; A61B 1/005

(52) U.S. Cl. CPC A61B 1/008; A61B 1/00149; A61B 1/0057

(57) ABSTRACT

An articulating flexible endoscope is provided. The articulating flexible endoscope comprises: an elongated member extending between a distal end and a proximal end, where the elongated member comprises a bending section steerable by one or more pull wires. The bending section comprises at least an inner flexure placed inside an outer flexure. The inner flexure is formed with outer structures to provide structural support for the one or more pull wires upon assembly with the outer flexure.

### CROSS-REFERENCE

This application is a continuation of International Application No. PCT/US2023/079054, filed on Nov. 8, 2023, which claims priority to U.S. Provisional Patent Application No. 63/383,788, filed on Nov. 15, 2022, which is entirely incorporated herein by reference.

### BACKGROUND

Endoscopy procedures use an endoscope to examine the interior of a hollow organ or cavity of the body. Unlike many other medical imaging techniques, endoscopes are inserted into the organ directly. Flexible endoscope that can deliver instinctive steering and control is useful in diagnosing and treating diseases that are accessible through any natural orifice in the body. Depending on the clinical indication, the endoscope may be designated as bronchoscope, ureteroscope, colonoscope, gastroscope, ENT scope, and various others. For example, flexible bronchoscope may be used for lung cancer diagnosis and/or surgical treatment. However, one challenge in bronchoscopy is reaching the upper lobe of the lung while navigating through the airways. In another example, flexible endoscopy has been used to inspect and treat disorders of the gastrointestinal (GI) tract without the need for creating an opening on the patient's body. The endoscope is introduced via the mouth or anus into the upper or lower GI tracts respectively. A miniature camera at the distal end captures images of the GI wall that help the clinician in their diagnosis of the GI diseases. Simple surgical procedures (like polypectomy and biopsy) can be performed by introducing a flexible tool via a working channel to reach the site of interest at the distal end.

Endoscopes are traditionally made to be re-usable, which may require thorough cleaning, dis-infection, and/or sterilization after each procedure. In most cases, cleaning, dis-infection, and sterilization may be aggressive processes to kill germs and/or bacteria. Such procedures may also be harsh on the endoscopes themselves. Therefore, the designs of such re-usable endoscopes can often be complicated, especially to ensure that the endoscopes can survive such harsh cleaning, dis-infection, and sterilization protocols. Periodical maintenance and repairs for such re-usable endoscopes may often be needed.

Low cost, disposable medical devices designated for a single-use have become popular for instruments that are difficult to clean properly. Single-use, disposable devices may be packaged in sterile wrappers to avoid the risk of pathogenic cross-contamination of diseases such as HIV, hepatitis, and other pathogens. Hospitals generally prefer the convenience of single-use disposable products because they no longer have to be concerned with product age, overuse, breakage, malfunction, and sterilization. Traditional endoscopes often include a handle that operators use to maneuver the endoscope. For single-use endoscopes, the handle usually encloses the camera, expensive electronics, and mechanical structures at proximal end in order to transmit the video and allow the users to maneuver the endoscope via a user interface. This may lead to high cost of the handle for a single-use endoscope.

### SUMMARY

Recognized herein is a need for a robotic endoscope that allows for performing surgical procedures or diagnostic operations with improved performance and cost-efficiency. Recognized also herein are devices and systems comprising endoscopes which may be disposable and may not require extensive cleaning procedures. The present disclosure provides a flexible endoscope with improved performance at reduced cost. In some embodiments, low-cost, single-use articulatable endoscope for diagnosis and treatment is provided and can be used in various applications such as bronchoscopy, urology, gynecology, arthroscopy, orthopedics, ENT, gastro-intestine endoscopy, neurosurgery, and various others. In some cases, the present disclosure provides a single-use, disposable, robotically controlled bronchoscope for use with a robotic system to enable diagnostic evaluation of lesions anywhere in the pulmonary anatomy. It should be noted that the provided endoscope systems can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others.

The endoscope herein may be an elongate device comprising a flexible elongate member. As sections of elongate device are curved as they are moved through the one or more passageways, the curved sections are subject to compression. The compression causes the elongate device to bend and contact the walls of one or more passageways along its length. In areas where there are one or more large passageways around the elongate device, when the distal end of the device encounters tissue resistance, the elongate device may bend within the large airway and move towards an unintended region of anatomy and not in the intended direction (“prolapsing”). Prolapsing is more probable when tissue resistance gets higher (e.g., due to airways being too small, lung not fully inflated, diseased tissue, blind insertion, etc.) The prolapse or kink may result in potential damage as it may expose the sharp edges of the kinked elongate device and complicate the surgical procedure. Moreover, a bent or kinked elongate device may render the system losing location/shape control of the device and it may block the passage of an instrument. Furthermore, a device that prolapses or kinks may not be able to provide adequate reach towards the target anatomy for performing the intended task. Recognized herein is a need for a steerable bending section of an endoscope with improved stability thereby preventing kinking.

In an aspect of the present disclosure, an improved bending section is provided for an articulatable flexible endoscope. The bending section may be articulated into one or more directions to control a direction of the endoscope. In some embodiments, the bending section may comprise axially or radially stacked composite components allowing for the flexibility to create different mechanical properties in torsion, tension, compression, and bending. In some embodiments, the bending section may be a composite bending section with an inner insert and an outer flexure and may create different mechanical properties in torsion, tension, compression, and bending. The bending section may be articulated by one or more pull wires. The inner flexure is formed with outer structures to provide structural support for the one or more pull wires upon assembly with the outer flexure. In other embodiments, the bending section may be a single composite bending section with integrated backbone structure that provides improved hoop strength and kink resistance. For example, a plurality of backbone components may be integrated into a polymer segment to vary a mechanical property of the bending section. The provided bending section can be easier to manufacture compared to existing solutions thereby reducing the cost in manufacturing.

In an aspect of the present disclosure, an articulatable flexible endoscope is provided. The endoscope comprises: an elongated member extending between a distal end and a proximal end and a distal tip portion connected to the distal end of the elongate member. The elongated member comprises a bending section steerable by one or more pull wires. The bending section comprises at least an inner flexure placed inside an outer flexure and the inner flexure is formed with structures to provide structural support for the one or more pull wires upon assembly with the outer flexure.

In some embodiments, the inner flexure comprises repeated cut pattern at a first predetermined longitudinal pitch. In some cases, the outer flexure comprises repeated cut pattern at a second predetermined longitudinal pitch. In some instances, the second predetermined longitudinal pitch is a multiplication of the first predetermined longitudinal pitch.

In some embodiments, the structures of the inner flexure comprise circumferential grooves and protrusions to form one or more lumens upon assembly with the outer flexure. In some cases, the distal tip portion comprises a structure to receive an imaging device, a position sensor, and an illumination device. In some instances, the one or more lumens provide structural support to one or more cables connected to the imaging device, the position sensor or the illumination device. In some instances, the distal tip portion has one or more chamfered surfaces. For example, the one or more chamfered surfaces are located substantially at a working channel. In some cases, the distal tip portion has a feature mating with a complementary feature of the bending section thereby providing a rotational alignment and lateral alignment. In some cases, the position sensor comprises a pair of electromagnetic sensors arranged at an angle.

In some embodiments, the inner flexure is formed of a plurality of modular components connected via interlocking features. In some embodiments, the proximal end is connected to a proximal portion comprising a driving mechanism for applying a force to the one or more pull wires. In some embodiments, the proximal portion is supported by a robotic arm.

In another aspect, an articulating flexible endoscope is provided. The endoscope comprises: an elongated member extending between a distal end and a proximal end, the elongated member comprises a bending section steerable by one or more pull wires, and a distal tip portion connected to the distal end of the elongate member. The bending section comprises a plurality of backbone components integrated into a plurality of polymer segments at various locations along the elongated member to vary a mechanical property of the bending section.

In some embodiments, the plurality of backbone components are thin disks formed of a material stiffer than a polymeric material. In some embodiments, the plurality of polymer segments have different lengths. In some cases, the different lengths are selected to adjust a minimum bending radius of the bending section.

In some embodiments, the plurality of polymer segments have different configuration of lumens formed in each of the plurality of polymer segments. In some embodiments, at least one of the plurality of backbone components is fused into at least one of the plurality of polymer segments. In some embodiments, each of the plurality of backbone components comprise one or more holes matching at least a portion of lumens formed in the plurality of polymer segments. In some cases, the distal tip portion comprises a structure to receive an imaging device, a position sensor, and an illumination device. In some instances, the lumens and holes provide structural support to one or more cables connected to the imaging device, the position sensor or the illumination device.

In some embodiments, the distal tip portion has one or more chamfered surfaces. In some cases, the one or more chamfered surfaces are located substantially at a working channel. In some instances, the position sensor comprises a pair of electromagnetic sensors arranged at an angle.

In some embodiments, the proximal end is connected to a proximal portion comprising a driving mechanism for applying a force to the one or more pull wires. In some cases, the proximal portion is supported by a robotic arm.

It should be noted that the provided modular endoscope components and various components of the device can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a bending section, in accordance with some embodiments of the present disclosure.

FIG. 2 schematically shows an example of a conventional bending section and an example of the bending section with backbone component(s).

FIG. 3 shows examples of a polymer segment and a backbone component.

FIG. 4 shows various examples of a polymer segment.

FIG. 5 shows an example of bending section with one or more additional elements.

FIG. 6 shows an assembled view and an exploded view of an example of a bending section.

FIG. 7 shows different views of a bending section comprising an inner flexure and an outer flexure.

FIG. 8 shows examples of an inner flexure with various different configurations.

FIG. 9 shows an example of bending section with a jacket placed at the tip portion.

FIG. 10 illustrates an example of a flexible endoscope, in accordance with some embodiments of the present disclosure.

FIG. 11 shows an example of a robotic bronchoscope comprising a handle portion and a flexible elongate member.

FIG. 12 shows an example of an instrument driving mechanism providing mechanical interface to the handle portion of the robotic bronchoscope.

FIG. 13 shows an example of a distal tip of an endoscope.

FIG. 14 shows an exemplary distal portion of the catheter with integrated imaging device and the illumination device.

FIGS. 15-19 show an example of a tip portion with a reduced diameter.

FIG. 20 shows an example of arrangement of a pair of electromagnetic (EM) sensors.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The embodiments disclosed herein can be combined in one or more of many ways to provide improved diagnosis and therapy to a patient. The disclosed embodiments can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

While exemplary embodiments will be primarily directed at a device or system for bronchoscopy, one of skill in the art will appreciate that this is not intended to be limiting, and the devices described herein may be used for other therapeutic or diagnostic procedures and in various anatomical regions of a patient's body. The provided device or system can be utilized in urology, gynecology, rhinology, otology, laryngoscopy, gastroenterology with the endoscopes, combined devices including endoscope and instruments, endoscopes with localization functions, one of skill in the art will appreciate that this is not intended to be limiting, and the devices described herein may be used for other therapeutic or diagnostic procedures and in other anatomical regions of a patient's body, such as such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat, and various others, in the forms of: NeuroendoScope, EncephaloScope, OphthalmoScope, OtoScope, RhinoScope, LaryngoScope, GastroScope, EsophagoScope, BronchoScope, ThoracoScope, PleuroScope, AngioScope, MediastinoScope, NephroScope, GastroScope, DuodenoScope, CholeodoScope, CholangioScope, LaparoScope, AmioScope, UreteroScope, HysteroScope, CystoScope, ProctoScope, ColonoScope, ArthroScope, SialendoScope, Orthopedic Endoscopes, and others, in combination with various tools or instruments.

The systems and apparatuses herein can be combined in one or more of many ways to provide improved diagnosis and therapy to a patient. Systems and apparatuses provided herein can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the terms distal and proximal may generally refer to locations referenced from the apparatus, and can be opposite of anatomical references. For example, a distal location of a primary shaft or catheter may correspond to a proximal location of an elongate member of the patient, and a proximal location of the primary sheath or catheter may correspond to a distal location of the elongate member of the patient.

As described above, one challenge in endoscopy is being flexible enough to navigate through tortuous pathways while being able to reach hard-to-reach body cavities and conduits (e.g., the upper lobe of the lung while navigating through the airway) without kink/prolapse.

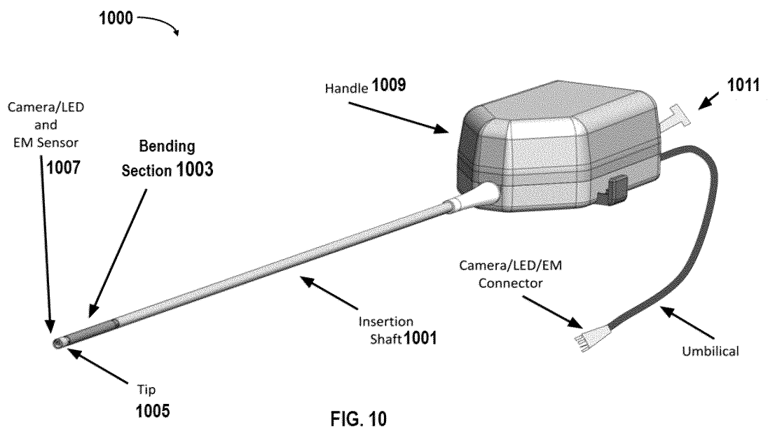

In an aspect of the present disclosure, a flexible endoscope with improved performance at reduced cost is provided. In some embodiments, a flexible elongate member of the endoscope herein may comprise a bending section with integrated backbone components. FIG. 10 illustrates an example of a flexible endoscope 1000, in accordance with some embodiments of the present disclosure. As shown in FIG. 10, the flexible endoscope may comprise a handle/proximal portion 1009 and a flexible elongate member to be inserted inside of a subject. In some embodiments, the flexible elongate member may comprise a proximal shaft (e.g., insertion shaft 1001), steerable tip (e.g., tip 1005), and a steerable section (bending section 1003). The bending section 1003 is located between the distal tip 1005 and insertion shaft 1001. The bending section may comprise configurations as described later herein. For example, the bending section may comprise a plurality of backbone components integrated into a polymer segment to vary a mechanical property of the bending section. Alternatively, the bending section may comprise radially stacked composite configuration. The endoscope 1000 may also be referred to as steerable catheter assembly as described elsewhere herein. In some cases, the endoscope 1000 may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of. In some embodiment, the endoscope may contain varying levels of stiffness along the shaft, as to improve functional operation.

The endoscope or steerable catheter assembly 1000 may comprise a handle portion 1009 that may include one or more components configured to process image data, provide power, or establish communication with other external devices. For instance, the handle portion may include a circuitry and communication elements that enables electrical communication between the steerable catheter assembly 1000 and an instrument driving mechanism (not shown), and any other external system or devices. In another example, the handle portion 1009 may comprise circuitry elements such as power sources for powering the electronics (e.g., camera, electromagnetic sensor and LED lights) of the endoscope.

The one or more components located at the handle may be optimized such that expensive and complicated components may be allocated to the robotic support system, a hand-held controller or an instrument driving mechanism thereby reducing the cost and simplifying the design the disposable endoscope. The handle portion or proximal portion may provide an electrical and mechanical interface to allow for electrical communication and mechanical communication with the instrument driving mechanism. The instrument driving mechanism may comprise a set of motors that are actuated to rotationally drive a set of pull wires of the catheter. The handle portion of the catheter assembly may be mounted onto the instrument drive mechanism so that its pulley/capstans assemblies are driven by the set of motors. The number of pulleys may vary based on the pull wire configurations. In some cases, one, two, three, four, or more pull wires may be utilized for articulating the flexible endoscope or catheter.

The handle portion may be designed allowing the robotic endoscope (e.g., bronchoscope) to be disposable at reduced cost. For instance, classic manual and robotic endoscope may have a cable in the proximal end of the bronchoscope handle. The cable often includes illumination fibers, camera video cable, and other sensors fibers or cables such as electromagnetic (EM) sensors, or shape sensing fibers. Such complex cable can be expensive adding to the cost of the bronchoscope. The provided robotic endoscope may have an optimized design such that simplified structures and components can be employed while preserving the mechanical and electrical functionalities. In some cases, the handle portion of the robotic endoscope may employ a cable-free design while providing a mechanical/electrical interface to the catheter.

The electrical interface (e.g., printed circuit board) may allow image/video data and/or sensor data to be received by the communication module of the instrument driving mechanism and may be transmitted to other external devices/systems. In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

In some cases, the handle portion 1009 may comprise one or more mechanical control modules such as lure 1011 for interfacing the irrigation system/aspiration system. In some cases, the handle portion may include lever/knob for articulation control. Alternatively, the articulation control may be located at a separate controller attached to the handle portion via the instrument driving mechanism.

The endoscope may be attached to a robotic support system or a hand-held controller via the instrument driving mechanism (IDM). The instrument driving mechanism may be provided by any suitable controller device (e.g., hand-held controller) that may or may not include a robotic system. The instrument driving mechanism may provide mechanical and electrical interface to the steerable catheter assembly 1000. The mechanical interface may allow the steerable catheter assembly 1000 to be releasably coupled to the instrument driving mechanism. For instance, the handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool.

In the illustrated example, the distal tip of the catheter or endoscope shaft is configured to be articulated/bent in two or more degrees of freedom to provide a desired camera view or control the direction of the endoscope. As illustrated in the example, imaging device (e.g., camera), position sensors (e.g., electromagnetic sensor) 1007 is located at the tip of the catheter or endoscope shaft 1005. For example, line of sight of the camera may be controlled by controlling the articulation of the bending section 1003. In some instances, the angle of the camera may be adjustable such that the line of sight can be adjusted without or in addition to articulating the distal tip of the catheter or endoscope shaft. For example, the camera may be oriented at an angle (e.g., tilt) with respect to the axial direction of the tip of the endoscope with aid of an optimal component.

The distal tip 1005 may be a rigid component that allow for positioning sensors such as electromagnetic (EM) sensors, imaging devices (e.g., camera) and other electronic components (e.g., LED light source) being embedded at the distal tip.

In real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. For example, the EM field generator may be positioned close to the patient torso during procedure to locate the EM sensor position in 3D space or may locate the EM sensor position and orientation in 5DOF (degrees of freedom) or 6DOF (degrees of freedom). This may provide a visual guide to an operator when driving the endoscope towards the target site.

The endoscope may have a unique design in the elongate member. In some embodiments, the bending section 1003 may comprise axially or radially stacked components as described elsewhere herein with improved mechanical properties. Details about the bending section are described later herein. In some cases, the proximal shaft of the endoscope may consist of a single tube that incorporates a series of cuts (e.g., reliefs, slits, etc.) along its length to allow for improved flexibility, and a desirable stiffness.

As described above, the bending section 1003 may be designed to allow for bending in two or more degrees of freedom (e.g., articulation). For example, the bending section may be steered or articulated in up, down, pitch, yaw direction, or any direction in-between. A greater bending degree such as 180 and 270 degrees (or other articulation parameters for clinical indications) can be achieved by the unique structure of the bending section while kinking or prolapse may be prevented. In some cases, the bending section may be fabricated separately as a modular component and assembled to the proximal shaft.

The articulation of the endoscope may be controlled by applying force to the distal end of the endoscope via one or multiple pull wires. The one or more pull wires may be attached to the distal end of the endoscope. In the case of multiple pull wires, pulling one wire at a time may change the orientation of the distal tip to pitch up, down, left, right or any direction needed. In some cases, the pull wires may be anchored at the distal tip of the endoscope, running through the bending section, and entering the handle where they are coupled to a driving component (e.g., pulley). This handle pulley may interact with an output shaft from the robotic system.

In some embodiments, the proximal end or portion of one or more pull wires may be operatively coupled to various mechanisms (e.g., gears, pulleys, capstans, etc.) in the handle portion of the catheter assembly. The pull wire may be a metallic wire, cable or thread, or it may be a polymeric wire, cable or thread. The pull wire can also be made of natural or organic materials or fibers. The pull wire can be any type of suitable wire, cable or thread capable of supporting various kinds of loads without deformation, significant deformation, or breakage. The distal end/portion of one or more pull wires may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The pull wires may be made of any suitable material such as stainless steel (e.g., SS316), metals, alloys, polymers, nylons or biocompatible material. Pull wires may be a wire, cable or a thread. In some embodiments, different pull wires may be made of different materials for varying the load bearing capabilities of the pull wires. In some embodiments, different sections of the pull wires may be made of different material to vary the stiffness and/or load bearing along the pull. In some embodiments, pull wires may be utilized for the transfer of electrical signals.

The proximal design may improve the reliability of the device without introducing extra cost allowing for a low-cost single-use endoscope. In some cases, the device may be a single-use robotic endoscope. The robotic endoscope (e.g., bronchoscope) and can be the same as the steerable catheter assembly as described elsewhere herein. Traditional endoscopes can be complex in design and are usually designed to be re-used after procedures, which require thorough cleaning, dis-infection, or sterilization after each procedure. The existing endoscopes are often designed with complex structures to ensure the endoscopes can endure the cleaning, dis-infection, and sterilization processes. The provided robotic endoscope (e.g., bronchoscope) can be a single-use endoscope that may beneficially reduce cross-contamination between patients and infections. In some cases, the robotic endoscope may be delivered to the medical practitioner in a pre-sterilized package and are intended to be disposed of after a single-use.

### Axially Stacked Bending Section

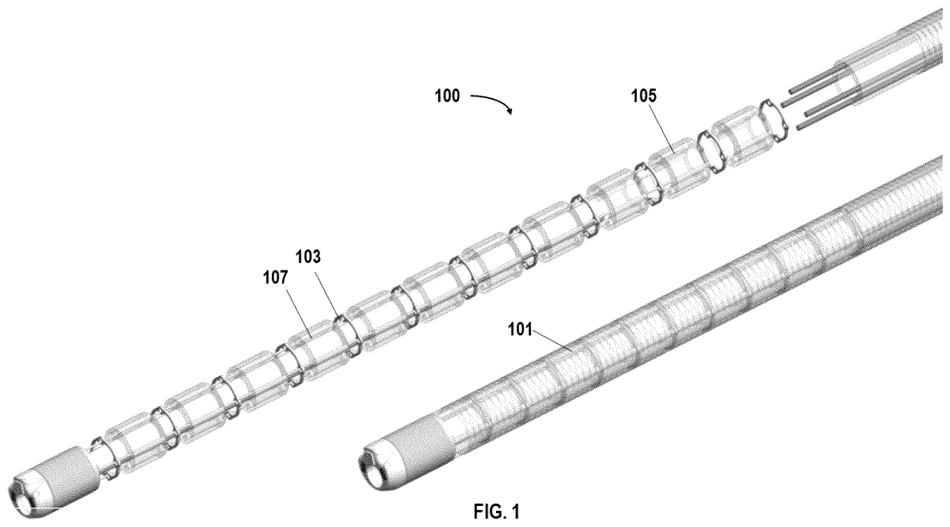

FIG. 1 shows an example of a bending section 100, in accordance with some embodiments of the present disclosure. In some embodiments, the bending section 100 may comprise a flexible component 101 formed of polymer material, biocompatible polymer material composite or elastomer material and the like integrated with a plurality of backbone components 103. In some cases, the materials of the flexible component 101 of the bending section may be selected such that it may maintain structural support to the internal structures (e.g., working channel) as well as being substantially flexible (e.g., able to bend in various directions and orientations). For example, the flexible component 101 can be made of any suitable material such as Provista Copolymer, vinyl (such as polyvinyl chloride), Nylon (such as vestamid, grilamid), pellethane, polyethylene, polyurethane, polypropylene, polycarbonate, polyester, silicon elastomer, acetate and so forth.

In some cases, the material of the flexible component of the bending section 101 may be selected such that it can be fused or integrated with a plurality of backbone components 103 to form a substantially single-piece bending section. For instance, the bending section 101 may be formed by fusing a plurality of extruded elastomer/polymer segments 105, 107 together with the plurality of backbone components 103. The fusing can be achieved using various methods such as lamination, dip-casting, or insert molding. The plurality of backbone components 103 may be disposed at various locations along the length of the bending section. In some cases, a bending section 100 may be formed by fusing a plurality of elastomer/polymer segments 105, 107 with one or more backbone component 103 disposed in between the elastomer/polymer segments to form a single-piece bending section 101.

In some embodiments, the plurality of elastomer/polymer segments 105, 107 may comprise a substantially tubular shape with internal structures. The internal structures may include, for example, a working channel for passing through tools, cavities or lumens for routing electrical wires, embedded sensors, or other components. Details about the structures of the elastomer/polymer segments are described later herein.

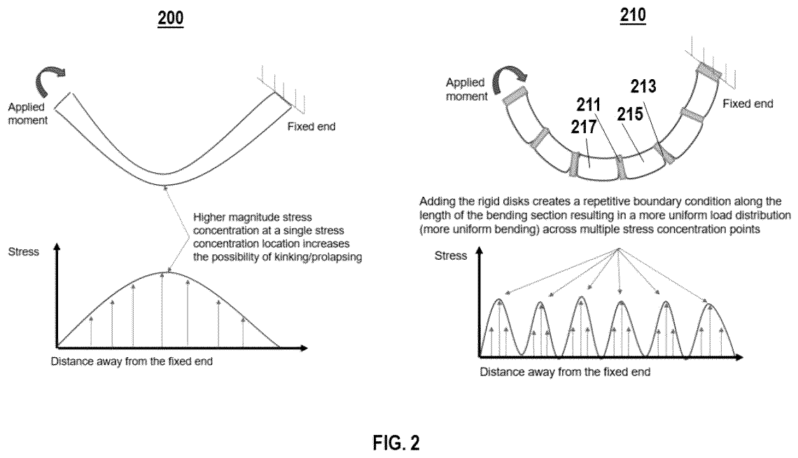

The plurality of backbone components may beneficially provide superior hoop strength and kink resistance compared to conventional catheter bending sections. FIG. 2 schematically shows an example 200 of the conventional bending section without backbone components and an example 210 of a bending section with the backbone components. The conventional bending section 200 without the backbone components may be prone to kinking or prolapsing due to the higher magnitude stress concentration at a single stress concentration location. In contrast, a bending section 210 with a plurality of backbone components 211, 213 may distribute the load/stress across the axial length of the bending section thereby reducing kinking or prolapsing. For instance, the backbone components may beneficially create repetitive boundary condition along with length of the bending section resulting in a relatively uniform load distribution (e.g., uniform bending) across the plurality of stress concentration points 211, 213. In some embodiments, the backbone components may comprise internal lumens for pull wires to pass through which help to translate tensions in the pull wires to bending motion of the bending section.

A bending section may comprise at least one, two, three, four, five, six, seven, or more backbone components 211, 213. The bending section may comprise at least one, two, three, four, five, six, seven, or more polymer sections 215, 217. The polymer sections 215, 217 can be the same as the elastomer/polymer segments as described above. In some embodiments, a bending configuration or bending geometries of the bending section may be selected by selecting the length of the polymer sections 215, 217 between each backbone components 211, 213, and/or the location of the backbone components along the axial length of the bending section. For instance, the plurality of backbone components can be placed at various locations along the length of the bending section so they may or may not be evenly distributed to achieve desirable mechanical properties (e.g., torsion, tension, compression, or bending) or a desirable bending configuration (e.g., minimum bending radius at a selected location along the length).

The backbone component may be formed of material (e.g., metal disk) that is generally stiffer or more rigid than the polymer section to reinforce the bending section at selected locations. In some cases, by selecting the location and/or number of backbone components in the bending section, a smaller minimum bend radius of bending section may be achieved to facilitate maneuverability of the elongated member, while preventing or reducing the likelihood of kinking or prolapsing. For example, by increasing the length of a polymer section between two backbone components at a location closer to the distal tip, a smaller minimum bend radius closer to the tip may be achieved thereby facilitating alignment of the tip with the airways in a tight space or with sharp turns. For example, the minimum bend radius of the bending section may be varied in a range of 5 mm-30 mm, a range of 8 mm-15 mm or any number below 5 mm or above 30 mm to allow the bending section to achieve articulation of at least 120-degree, 130-degree, 140-degree, 150-degree, 160-degree, 170-degree, 180-degree, 190-degree, 200-degree or greater.

In some cases, decreasing the length of a polymer section between two backbone components (e.g., at a location further away from the distal tip) may beneficially increase the stiffness thereby preventing prolapsing or kinking. For instance, by integrating more backbone components at locations further away from the distal tip may beneficially prevent the bending section to bend any further beyond a minimum bend radius. The flexibility of adjusting the bending configuration or geometrics of the bending section may allow for a bending section adaptive to a particular use application. For instance, to help to bridge the gap in the right main bronchus and/or prevent prolapsing, the length of one or more polymer sections in a portion of the bending section that is further away from the distal tip may be decreased such that a minimum bend radius of the portion may be in a range of 1 cm-3 cm or any number below 1 cm or greater than 3 cm. In some cases, the location and/or the length for the polymer section may be determined based on a curvature or size of the passageway inside a patient.

By varying a dimension, shape, structure, material, or other design of the polymer sections and/or backbone components, the device herein has the flexibility of adjusting mechanical properties (e.g., bending radius, friction, stiffness, or strength) at selected section or location along the bending section without introducing extra manufacturing cost. For example, by selecting different polymer segments of different lengths to fuse with backbone components, variable bending stiffness along the length of the bending section can be conveniently achieved. Furthermore, by selecting polymers of various stiffness in between the backbone components, various bending shapes can be achieved.

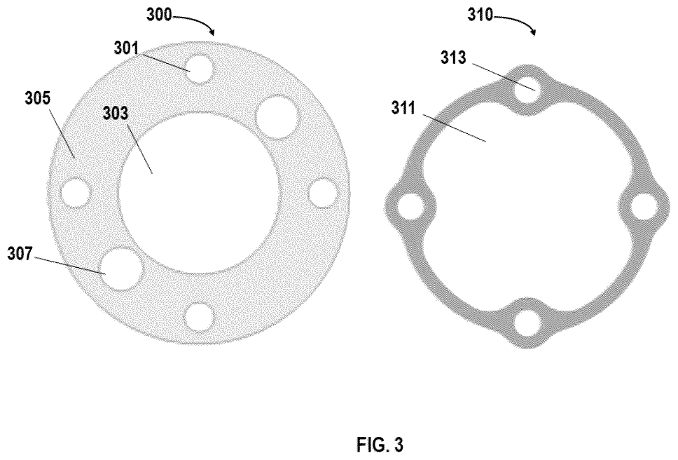

FIG. 3 shows examples of a polymer segment 300 and a backbone component 310. In some embodiments, the backbone component 310 may be formed of a material that is more rigid than the material of the polymer segment 300. For example, the material of the backbone component may be metallic materials such as stainless steel or nitinol, stiff polymers such as PEEK, glass or carbon filled PEEK, Ultem, Polysulfone, ceramics, composite material and other suitable materials. In some cases, the backbone component may be a metal disk that is chemically etched, laser cut, molded or stamped to form the shape, or profile.

In some cases, the backbone component 310 may have a thin or small thickness so it can be easily fused into the polymer segment such as during a lamination process. Once it is fused with the polymer segment, at least a portion of or the entirety of the backbone component may be surrounded by the polymer segment. In some cases, the backbone component may have an outer profile or an outer dimension (e.g., largest diameter) that is substantially the same or smaller than the outer diameter of the polymer segment 310 such that once the backbone component is fused into the polymer segment, the integrated single-piece bending section (e.g., 101 in FIG. 1) may have a smooth outer surface.

In some embodiments, the backbone component may have an inner profile that accommodates an inner profile of the polymer segment. For instance, the backbone component 310 may have a substantially circular shape that can be fit within a wall 305 of the polymer segment 300. The backbone component may comprise at least an inner profile 311 that can accommodate the lumen 303 of the polymer segment. For example, a dimension (e.g., smallest diameter) of the inner profile is no smaller than a diameter of the lumen of the polymer segment. In some cases, the backbone component may also comprise one or more holes 313 to accommodate one or more holes 301 of the polymer segment. For example, once the backbone component and the polymer segment are aligned by the center, at least one or more inner structures of the backbone component 313 can be aligned with one or more inner structures 301 of the polymer segment for passing through one or more pull wires. The inner structures 311, 303 may also provide passage or lumens for routing the electrical wires or housing other components inside the endoscope device.

In some embodiments, the inner profile of the polymer segment 300 may comprise at least a lumen 303 for the working channel, and one or more lumens 301 for passing through one or more pull wires. In optional embodiments, the polymer segment 300 may comprise additional one or more lumens 307 for routing one or more electrical wires (e.g., cables to supply powers to the sensor, LED, camera, etc. located at the distal tip). In some cases, the one or more lumens 307 for routing one or more electrical wires may have a diameter greater than that of the lumens for the one or more pull wires resulting a thicker wall around the one or more lumens 301. In some cases, the wall thickness around the one or more lumens for accommodating the one or more pull wires may be increased to balance the forces around the neutral axis. In some cases, the backbone component may have an inner profile or inner structures that may not match exactly the lumens formed in the wall of the polymer segment. For instance, the one or more lumens 301 for the one or more pull wires may but with the one or more inner structures 313 of the backbone component and the backbone component may not have separate holes corresponding to the one or more lumens 307 for the electric wires. Alternatively, the backbone component may have inner profile or inner structures that match the lumens formed in the wall of the polymer segment.

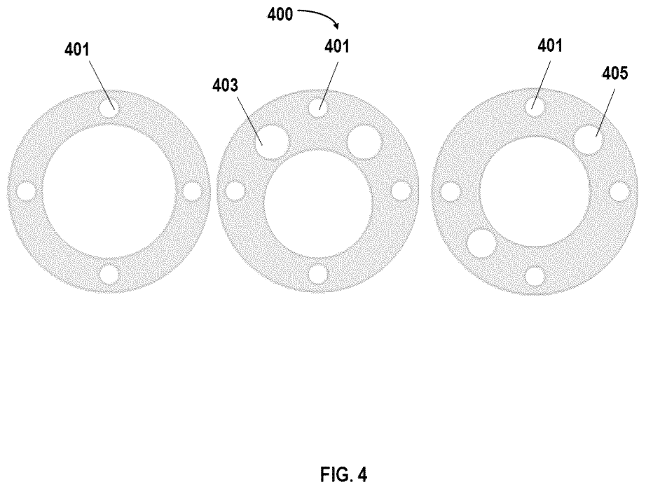

Depending on the requirement, the polymer segments may or may not have the additional lumens for the electric wires. The polymer segments may have various different inner profiles or configurations. FIG. 4 shows various examples 400 of the polymer segments. As shown in the example 401, the polymer segment may have a lumen in the center for working channel and a plurality of lumens 401 in the wall for accommodating a plurality of pull wires. In a different example, the polymer segment may have additional lumens 403 for one or more electric wires. The additional lumens 403 may or may not have the same dimension (e.g., diameter) as the lumens 401 for the pull wires. The additional lumens 403, 405 may be located in various locations relative to the lumens 401 for the pull wires. In some cases, the lumen configuration such as location and/or dimension (e.g., diameter) of the lumens 403, 405 may be varied to achieve desirable bending configuration or stiffness.

In some embodiments, the bending section may comprise a plurality of polymer segments having identical inner profiles and/or outer profiles. Alternatively, the plurality of individual polymer segments to form a single bending section may have different inner profiles, and/or lumen configurations. For example, polymer segments with different length, material, inner profiles, lumen configurations and the like may be selected to form a bending section. As described above, the polymer segments may be formed of polymer, elastomer or any suitable material such as polyethylene terephthalate (PET), PTFE, pebax, polyurethane, polyamide (PA), or nylon that may be less stiffer than the material of the backbone component. In some cases, an individual polymer segment may be manufactured by polymer extrusion, molding or other suitable manufacturing methods.

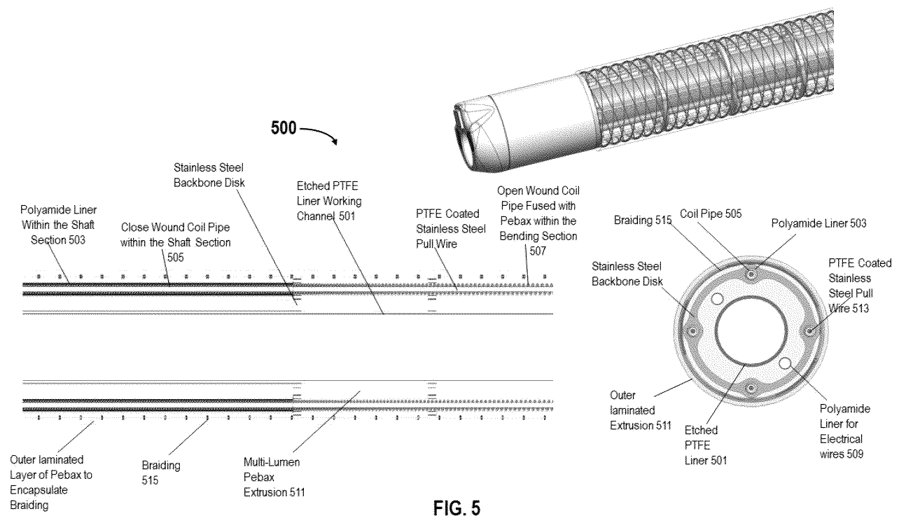

In some cases, the bending section may comprise one or more additional elements for further improving the performance. For example, the bending section may be laminated in layers to include liners that reduce friction within the catheter lumens. In some cases, the bending section may comprise coils and braids for added kink resistance and stiffness. In some cases, the bending section may comprise coil pipes to reinforce the pull wire path within the bending section and resolve the unintended motion of the shaft during articulation. FIG. 5 shows an example of bending section 500 integrated with one or more additional elements. In the illustrated example, the additional elements may include an etched PTFE liner 501, polyamide liners 503, coil pipes with close wound coils 505 inside a polyamide tube within the shaft section, or open wound coil popes 507 within the bending section.

In the illustrated example, the polymer section or polymer segment 511 may be integrated with one or more additional elements. For example, the polymer segment (e.g., polymer extrusions 511) may get stacked and laminated in a single or multiple steps along with the internal coil pipes, backbone disks, liners, pull wires 513, braiding 515, and the like. The polyamide lumens 509 may provide a path for routing electrical wires from the distal end of the catheter to the proximal end. The etched PTFE liner 501 provides a path for passing instruments through the catheter. These different lumens can vary in size, number, or shapes depending on the end effectors, sensors, or instrument needs at the distal end. The wound portion of the coil pipe within the shaft in the illustrated example may slide freely within the polyamide tube surrounding it and be fixed at the proximal end of the bending section as well as the proximal end of the shaft to resolve the unintended motion of the shaft during articulation. The unwound portion of the coil pipe within the bending section may be laminated to be fused within the polymer extrusions. The backbone disks within the bending section may beneficially provide hoop strength and structural support for the bending section while the pull wires are tensioned.

The braiding 515 fused with the polymers can have constant or variable pitch or braid patterns for additional torsional, bending, and axial stiffness. Instead of Pebax, other suitable materials such as polyurethane, or nylon compounds may be used. The stacks of polymers may have different stiffness ratings to achieve different bending shapes and variable stiffness profiles in bending, torsion, tension, or compression. Alternative liner materials such as PTFE doped polyamide tubes may be used instead of pure PTFE or polyamide. Instead of stainless steel, other suitable materials such as nitinol or tungsten may be used for the pull wires.

The stacked configuration may also allow for embedding or housing electronics, sensors (e.g., ultrasound probe, EM sensors, shape sensing fibers, etc.) and the like at different locations within the bending section. This may beneficially avoid interference with metals in the surroundings of the electronics.

### Radially Stacked Bending Section

In some embodiments, a bending section may be a composite with an inner flexure (e.g., insert) and an outer flexure. Such radially composition configuration may beneficially allow for different mechanical properties in torsion, tension, compression, and/or bending.

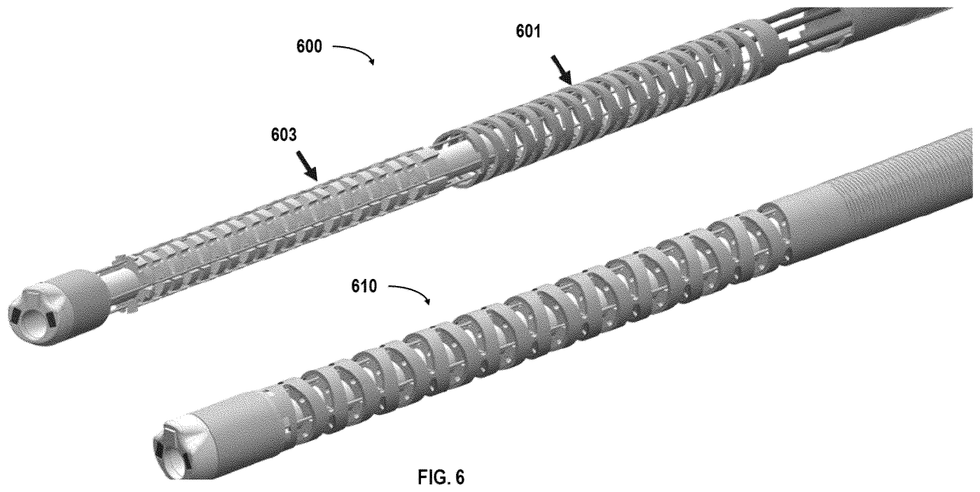

FIG. 6 shows an assembled view 610 and an exploded view 600 of an example of the bending section. As illustrated in the example, the bending section may comprise an outer flexure 601 and an inner flexure 603. In some cases, the outer flexure 601 may be a substantially tubular structure with cuts. In some cases, the cuts may be repeated pattern with a predetermined pitch. For example, the outer flexure may be monolithic laser cut stainless steel or nitinol hypotube with cuts that is laser cut or machined to form spring-like or link structures. In some cases, the inner flexure 603 may be single piece with cut patterns to form a flexure. The cut patterns may comprise repeated features ranged with a predetermined pitch. Alternatively, the inner flexure may comprise a stack of individual links that form a chain. In some cases, the inner flexure may comprise a lumen for the working channel, and structures such as open circumferential lumens for routing pull wires and/or electrical wires.

The inner flexure may be inserted inside the outer flexure to encapsulate one or more pull wires around the circumference of the inner flexure. Such radially composite configuration may beneficially reduce kinking or prolapsing by fully encapsulating the pull wires (e.g., hypotubes) within the bending section. The inner flexure may be fit within the outer flexure and once assembled, the inner and outer flexures may not move relative to one another.

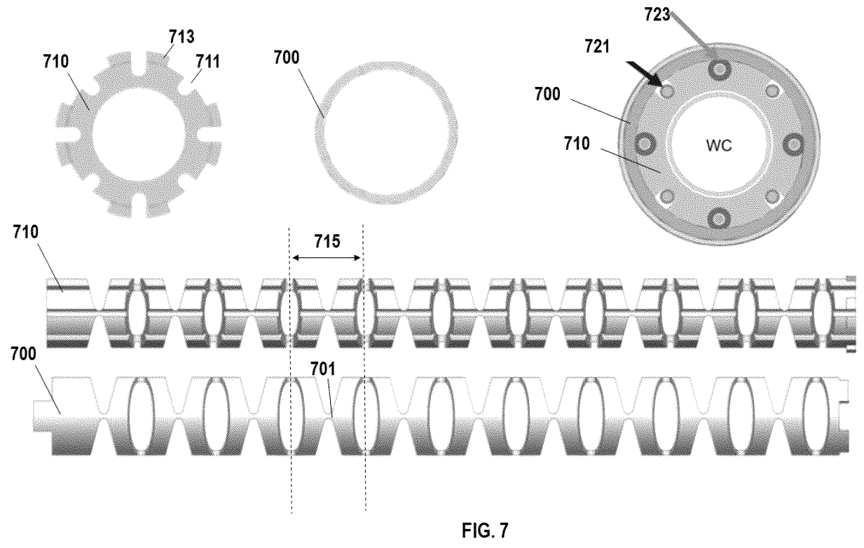

FIG. 7 shows different views of a bending section with an inner flexure 710 and an outer flexure 700. As shown in the cross-sectional view, the assembled bending section may comprise the inner flexure 710 inside of the outer flexure 700.

The inner flexure 710 may comprise outer features such as one or more grooves 711 and one or more protrusions 713 formed on the outer surface. Once the inner flexure is assembled with the outer flexure, such features 711, 713 may form one or more channels or lumens 723, 721 with an inner surface of the outer flexure for encapsulating or housing pull wires and electric wires. In some cases, such features (e.g., grooves, protrusions, tabs, counterbores, etc.) may have a dimension (e.g., width, diameter, depth) substantially match a dimension of the pull wires or other elements to be placed inside. For example, the dimension or size of the lumen 723 for accommodating the pull wire may be different from the size of the lumen 721 for an electric wire. In some cases, such features may be formed across the entire length of the bending section. Alternatively, such features may be formed only at the distal and/or proximal end of the bending section to align and mate with the outer flexure and capturing pull wire hypotubes or coil pipes. For example, no more than 10%, 20%, 30%, 40% of the entire length of the bending section may have such features for capturing the pull wires. Such structures of the inner flexure may allow for flexibility for placing electrical wires, working channel(s), coil pipes, pull wires or hypo tubes, electronics (e.g., ultrasound probe, EM sensors, shape sensing fibers, etc.), and/or other components that need to reside inside the bending section or pass through it.

As shown in the side view, the inner flexure 710 and an outer flexure 700 may have matching longitudinal flexure pitch 715. The longitudinal flexure pitch may refer to the pitch of the repeated cut pattern of the inner flexure or the outer flexure. In some cases, the flexure pitch of inner flexure 710 may be a multiplication (e.g., 1, 2, 3, 4, 5, etc.) of the pitch of the outer tube flexure. As shown in the example, the inner flexure 710 may be a single piece with cut patterns to form a flexure. The inner flexure 710 can be formed of any suitable material. For example, the material may be metallic materials such as stainless steel or nitinol, stiff polymers such as PEEK, glass or carbon filled PEEK, Ultem, Polysulfone and other suitable materials. In some cases, the material may be selected for easier manufacturing (e.g., laser cut pattern, extruded polymer, injection molded polymer, 3D printed polymer or metal, caster metal, etc.) thereby reducing the fabrication cost.

In some cases, the outer flexure 700 may provide improved hoop strength, torque transmission, and bend radius control compared to conventional bending section without a composite configuration. In some embodiments, the outer flexure may be a single piece flexure with spring-back-to-center properties. For example, the outer flexure may be formed of monolithic laser cut stainless steel or nitinol hypotube with suitable cut pattern to provide desirable spring-back-to-center property and/or bending stiffness. Alternatively, the outer flexure may not have spring-back-to-center properties. As shown in the side view, a hinge location or bend axes 701 may match that of the inner flexure. It should be noted that the cut pattern in the outer flexure and inner flexure may have various different sizes, shapes, pitches or location sections to achieve different mechanical properties (e.g., tortional stiffness, bending stiffness, bending radius, etc.). In some cases, the cut pattern may vary in pitch, size, shape (e.g., gap size or shape), location/distribution along the length of the bending section to achieve variable mechanical properties (e.g., tortional stiffness, bending stiffness, bending radius, etc.) along the length of the bending section. For example, the pitch may not be uniform along the length as a smaller pitch may beneficially prevent kinking of the pull wire when the pull wire is placed inside of the lumen structures 723.

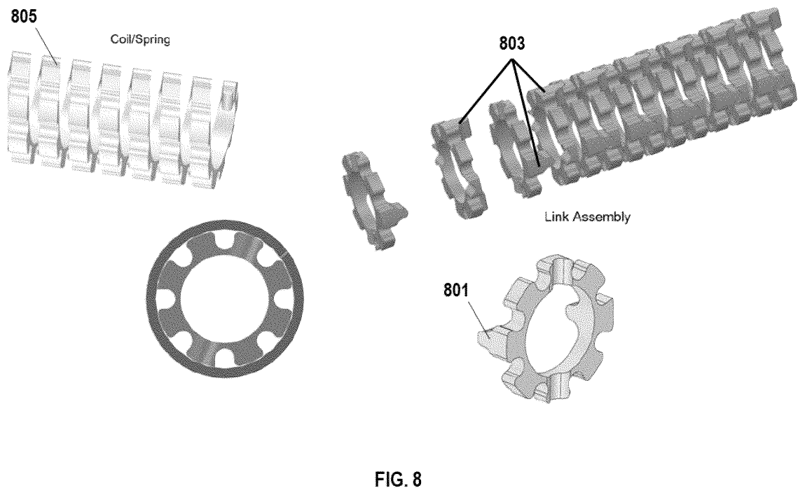

In some embodiments, the inner flexure and/or the outer flexure may be a one-piece flexure. Alternatively, the inner flexure and/or the outer flexure may comprise a stack of linked components. FIG. 8 shows examples of the inner flexure with different configurations. In some cases, the inner flexure may comprise a plurality of modular components 803 with interlocking features 801. The plurality of modular components 801 may be linked or coupled to each other via the interlocking features 801 without additional fasten means. In some cases, the plurality of modular components may be coupled or decoupled without additional tools. Alternatively, additional coupling means such as adhesive or welding may be used to connect the modular components. The interlocking features may beneficially provide axial and rotational alignment. The modular and interlocking features may allow for flexibility to vary the structures of the modular components thereby varying the mechanical properties of the bending section. For example, the plurality of modular components with different structures or mechanical properties (e.g., material, bending stiffness, wall thickness, etc.) may be selected to form a chain such that by placing a given modular component at a selected location along the length of the elongated member, the mechanical property of the bending section can be modified. In some cases, the connecting point or the location of the interlocking features 803 along the circumference may be selected or varied for connecting two modular components such that a chain of linked modular components may form a substantially coil spring structure 805.

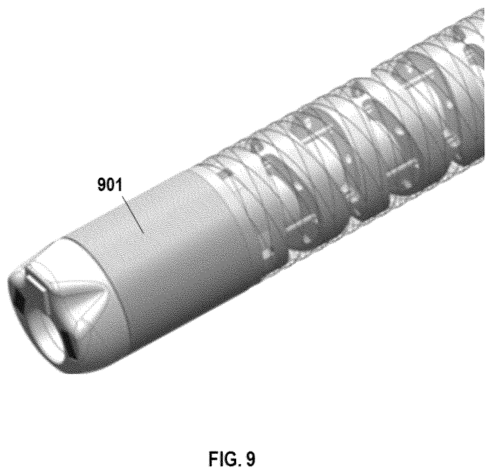

In some cases, the bending section may include additional elements such as coils, coil pipes, springs, braiding and the like to increase the strength, adjust the strength or modify the stiffness profiles of the bending section. In some cases, the bending section may include elements such as liners, laminations, jacket and the like to reduce friction. FIG. 9 shows an example of bending section with a jacket 901 placed at the tip portion. The jacket can be the same as the jacket as described above. For example, the jacket may slip over the distal portion the bending section to reduce the friction. The jacket may be formed of polymer or any suitable material such as PTFE, pebax, polyurethane, or nylon. In some cases, a dimension, materials and/or features forming the jacket may be selected to provide a smooth outer layer and/or variable stiffness.

### Flexible Endoscope

In some embodiments, the bending section herein may be utilized for improving flexibility and stability of a flexible endoscope without introducing extra cost. The provided bending section may be utilized by any devices or apparatuses. In an aspect of the invention, a flexible endoscope with improved performance (e.g., improved reliability) at reduced cost is provided. FIG. 10 illustrates an example of a flexible endoscope 1000, in accordance with some embodiments of the present disclosure. As shown in FIG. 10, the flexible endoscope 1000 may comprise a handle/proximal portion 1009 and a flexible elongate member to be inserted inside of a subject. The flexible elongate member can be the same as the one described above. In some embodiments, the flexible elongate member may comprise a proximal shaft (e.g., insertion shaft 1001), steerable tip (e.g., tip 1005), and a steerable section (bending section 1003). The bending section may have improved performance. For instance, the bending section may comprise radially composite configuration or axially stacked configuration as described above.

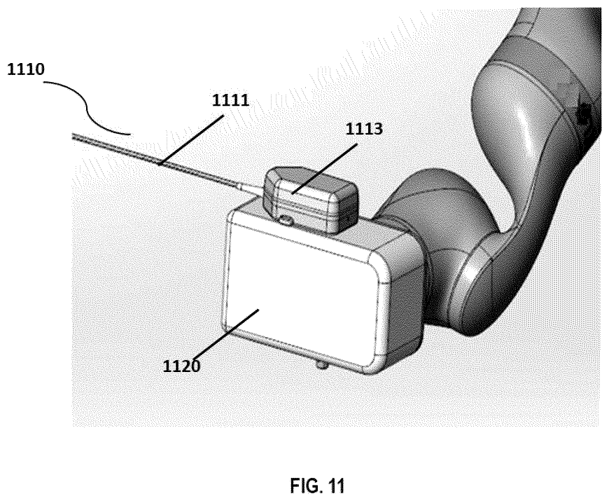

As shown in FIG. 11, a robotic endoscope (e.g., bronchoscope) 1120 may comprise a handle portion 1113 and a flexible elongate member 1111. In some embodiments, the flexible elongate member 1111 may comprise a shaft, steerable tip, a steerable bending section and an anti-prolapse passive section. The robotic endoscope 1120 can be the same as the steerable catheter assembly as described in FIG. 10. The robotic endoscope may be a single-use robotic endoscope. In some cases, only the catheter may be disposable. In some cases, at least a portion of the catheter may be disposable. In some cases, the entire robotic endoscope may be released from the instrument driving mechanism and can be disposed of. In some cases, the endoscope may contain varying levels of stiffness along its shaft, as to improve functional operation. In some cases, a minimum bend radius along the shaft may vary so that the kink resistance capability may be configurable along the length.

The robotic endoscope can be releasably coupled to an instrument driving mechanism 1120. The instrument driving mechanism 1120 may be mounted to the arm of the robotic support system or to any actuated support system as described elsewhere herein. The instrument driving mechanism may provide mechanical and electrical interface to the robotic endoscope 1110. The mechanical interface may allow the robotic endoscope 1110 to be releasably coupled to the instrument driving mechanism. For instance, the handle portion of the robotic endoscope can be attached to the instrument driving mechanism via quick install/release means, such as magnets and spring-loaded levels. In some cases, the robotic endoscope may be coupled or released from the instrument driving mechanism manually without using a tool.

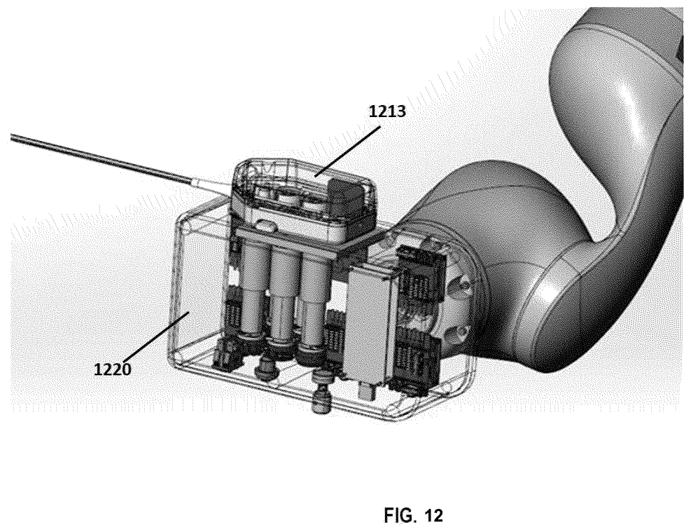

FIG. 12 shows an example of an instrument driving mechanism 1220 providing mechanical interface to the handle portion 1213 of the robotic endoscope. As shown in the example, the instrument driving mechanism 1220 may comprise a set of motors that are actuated to rotationally drive a set of pull wires of the flexible endoscope or catheter. The handle portion 1213 of the catheter assembly may be mounted onto the instrument drive mechanism so that its pulley assemblies or capstans are driven by the set of motors. The number of pulleys may vary based on the pull wire configurations. In some cases, one, two, three, four, or more pull wires may be utilized for articulating the flexible endoscope or catheter.

The handle portion may be designed allowing the robotic endoscope to be disposable at reduced cost. For instance, classic manual and robotic bronchoscopes may have a cable in the proximal end of the bronchoscope handle. The cable often includes illumination fibers, camera video cable, and other sensors fibers or cables such as electromagnetic (EM) sensors, or shape sensing fibers. Such complex cable can be expensive, adding to the cost of the endoscope. The provided robotic endoscope may have an optimized design such that simplified structures and components can be employed while preserving the mechanical and electrical functionalities. In some cases, the handle portion of the robotic bronchoscope may employ a cable-free design while providing a mechanical/electrical interface to the catheter.

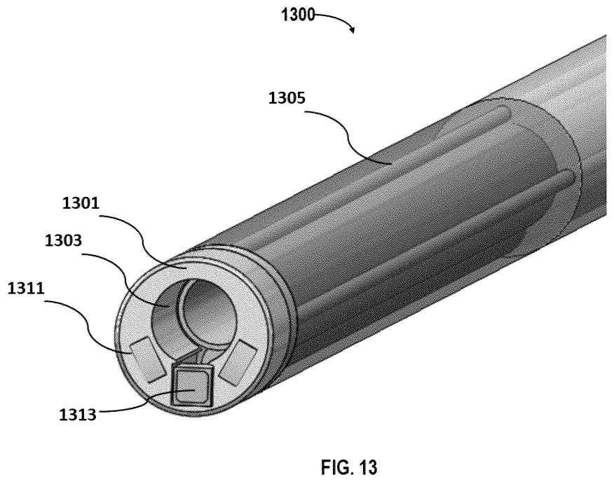

FIG. 13 shows an example of a distal tip 1300 of an endoscope. In some cases, the distal portion or tip of the catheter 1300 may be substantially flexible such that it can be steered into one or more directions (e.g., pitch, yaw). The catheter may comprise a tip portion, bending section, and insertion shaft as described above. In some embodiments, the catheter may have variable bending stiffness along the longitudinal axis direction. For instance, the catheter may comprise multiple sections having different bending stiffness (e.g., flexible, semi-rigid, and rigid). The bending stiffness may be varied by selecting materials with different stiffness/rigidity, varying structures in different segments (e.g., cuts, patterns), adding additional supporting components or any combination of the above. In some embodiments, the catheter may have variable minimum bend radius along the longitudinal axis direction. The selection of different minimum bend radius at different location long the catheter may beneficially provide anti-prolapse capability while still allow the catheter to reach hard-to-reach regions. In some cases, a proximal end of the catheter needs not be bent to a high degree thus the proximal portion of the catheter may be reinforced with additional mechanical structure (e.g., additional layers of materials) to achieve a greater bending stiffness. Such design may provide support and stability to the catheter. In some cases, the variable bending stiffness may be achieved by using different materials during extrusion of the catheter. This may advantageously allow for different stiffness levels along the shaft of the catheter in an extrusion manufacturing process without additional fastening or assembling of different materials.

The distal portion of the catheter may be steered by one or more pull wires 1305. The distal portion of the catheter may be made of any suitable material such as co-polymers, polymers, metals or alloys such that it can be bent by the pull wires. In some embodiments, the proximal end or terminal end of one or more pull wires 1305 may be coupled to a driving mechanism (e.g., gears, pulleys, capstan etc.) via the anchoring mechanism as described above.

The pull wire 1305 may be a metallic wire, cable or thread, or it may be a polymeric wire, cable or thread. The pull wire 1305 can also be made of natural or organic materials or fibers. The pull wire 1305 can be any type of suitable wire, cable or thread capable of supporting various kinds of loads without deformation, significant deformation, or breakage. The distal end or portion of one or more pull wires 1305 may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The catheter may have a dimension so that one or more electronic components can be integrated to the catheter. For example, the outer diameter of the distal tip may and the diameter of the working channel 1303 may be selected such that one or more electronic components can be embedded into the wall of the catheter. However, it should be noted that based on different applications, the outer diameter and the diameter of the working channel can be in any suitable range according to the tool dimensional or specific application.

The one or more electronic components may comprise an imaging device, illumination device or sensors. In some embodiments, the imaging device may be a video camera 1313. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

The illumination device may comprise one or more light sources 1311 positioned at the distal tip. The light source may be a light-emitting diode (LED), an organic LED (OLED), a quantum dot, or any other suitable light source. In some cases, the light source may be miniaturized LED for a compact design or Dual Tone Flash LED Lighting.

### Distal Portion or Tip of the Catheter

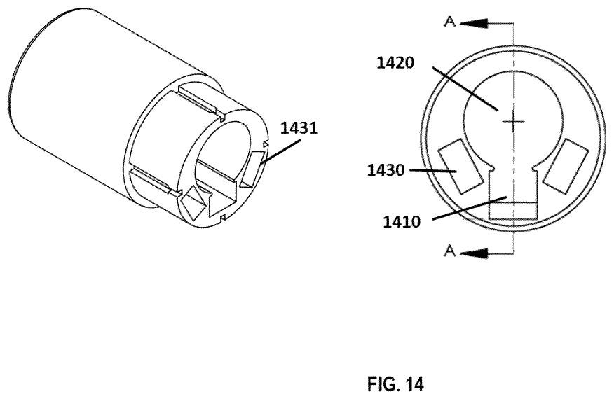

The imaging device and the illumination device may be integrated to the catheter. For example, the distal portion of the catheter may comprise suitable structures matching at least a dimension of the imaging device and the illumination device. The imaging device and the illumination device may be embedded into the catheter. FIG. 14 shows an example distal portion of the catheter with integrated imaging device and the illumination device. A camera may be located at the distal portion. The distal tip may have a structure to receive the camera, illumination device and/or the location sensor. For example, the camera may be embedded into a cavity 1410 at the distal tip of the catheter. The cavity 1410 may be integrally formed with the distal portion of the cavity and may have a dimension matching a length/width of the camera such that the camera may not move relative to the catheter. The camera may be adjacent to the working channel 1420 of the catheter to provide near field view of the tissue or the organs. In some cases, the attitude or orientation of the imaging device may be controlled by controlling a rotational movement (e.g., roll) of the catheter.

The power to the camera may be provided by a wired cable. In some cases, the cable wire may be in a wire bundle providing power to the camera as well as illumination elements or other circuitry at the distal tip of the catheter. The electric wires or cables may be captured by the internal structures of the bending section as described above. The camera and/or light source may be supplied with power from a power source located at the handle portion via wires, copper wires, or via any other suitable means running through the length of the catheter. In some cases, real-time images or video of the tissue or organ may be transmitted to an external user interface or display wirelessly. The wireless communication may be WiFi, Bluetooth, RF communication or other forms of communication. In some cases, images or videos captured by the camera may be broadcasted to a plurality of devices or systems. In some cases, image and/or video data from the camera may be transmitted down the length of the catheter to the processors situated in the handle portion via wires, copper wires, or via any other suitable means. The image or video data may be transmitted via the wireless communication component in the handle portion to an external device/system. In some cases, the system may be designed such that no wires are visible or exposed to operators.

In conventional endoscopy, illumination light may be provided by fiber cables that transfer the light of a light source located at the proximal end of the endoscope, to the distal end of the robotic endoscope. In some embodiments of the disclosure, one or more miniaturized LED lights may be employed and embedded into the distal portion of the catheter to reduce the design complexity. In some cases, the distal portion may comprise a structure 1430 having a dimension matching a dimension of a miniaturized LED light source. The distal portion may be embedded with one or more LED light sources. As shown in the illustrated example, two cavities 1430 may be integrally formed with the catheter to receive two LED light sources. For instance, the outer diameter of the distal tip may be around 4 to 4.4 millimeters (mm) and diameter of the working channel of the catheter may be around 2 mm such that two LED light sources may be embedded at the distal end. The outer diameter can be in any range smaller than 4 mm or greater than 4.4 mm, and the diameter of the working channel can be in any range according to the tool's dimensional or specific application. Any number of light sources may be included. The internal structure of the distal portion may be designed to fit any number of light sources.

In some cases, each of the one or more LEDs may be connected to power wires which may run to the proximal handle. In some embodiment, the one or more LEDs may be soldered to separated power wires that later bundle together to form a single strand. In some embodiments, the LEDs may be soldered to pull wires that supply power. In other embodiments, the one or more LEDs may be crimped or connected directly to a single pair of power wires. In some cases, a protection layer such as a thin layer of biocompatible glue may be applied to the front surface of the one or more LEDs to provide protection while allowing light emitted out. In some cases, an additional cover 1431 may be placed at the forwarding end face of the distal tip providing precise positioning of the one or more LEDs as well as sufficient room for the glue. The cover 1431 may be composed of transparent material matching the refractive index of the glue so that the illumination light may not be obstructed.

The working channel (e.g., working channel 1303, 1420) may be designed to provide protection for the internal components such as flexible instruments (e.g., needle, forceps, etc.). When flexible instruments pass through a conventional working channel, they may be abstracted by the working channel due to kinking, ovalizing and/or high friction force. The working channel herein may advantageously address the above drawbacks by providing a high hoop strength and a capability of achieving low bend radius. The working channel may also be designed to provide low friction in the inner surface.

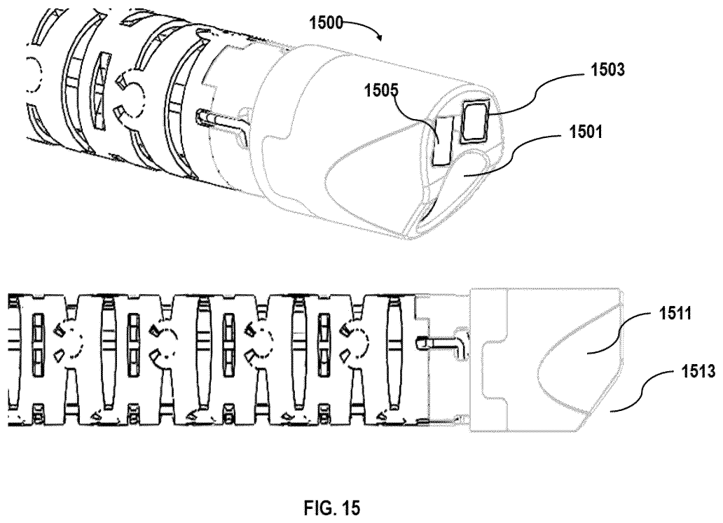

In some embodiments, the distal portion or the tip may have a reduced dimension to fit into an airway. FIGS. 15-20 show an example of a tip with a smaller diameter. As illustrated in FIG. 15, the distal portion 1500 may have an angled or chamfered tip so that the leading distal end of the tip has a smaller diameter. The angled or chamfered tip beneficially allows the endoscope to fit into smaller airways.

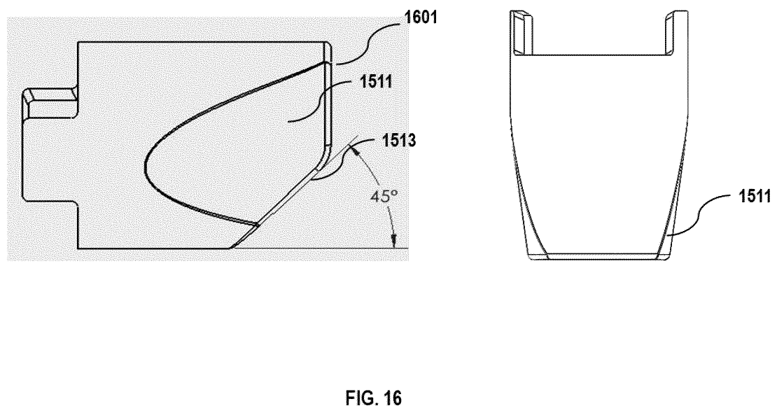

The angled tip may have one or more chamfered surfaces 1511, 1513. The chamfered surface may comprise a main chamfer 1513 located at the frontend surface of the distal tip. As further illustrated in FIG. 16, the angled tip may have a frontend surface formed of a main chamfer surface 1513 and a non-angled surface 1601. The main chamfer surface may have any suitable angle (e.g., about 45° angle, about 25° angle, about 30° angle, about 35° angle, about 40° angle, about 50° angle, about 55° angle, about 60° angle, about 65° angle, about 70° angle, etc.) to reduce the distal diameter of the tip. In some embodiments, the one or more chamfered surfaces may comprise one or more chamfers on the side (e.g., left or right side or both sides) which helps to further reduce the distal diameter of the tip 1511. The chamfers on the sides may have angles and dimensions suitable to reduce the diameter of the tip while allowing for sufficient internal structure to hold the one or more components embedded at the distal tip (e.g., camera, lighting device, etc.).

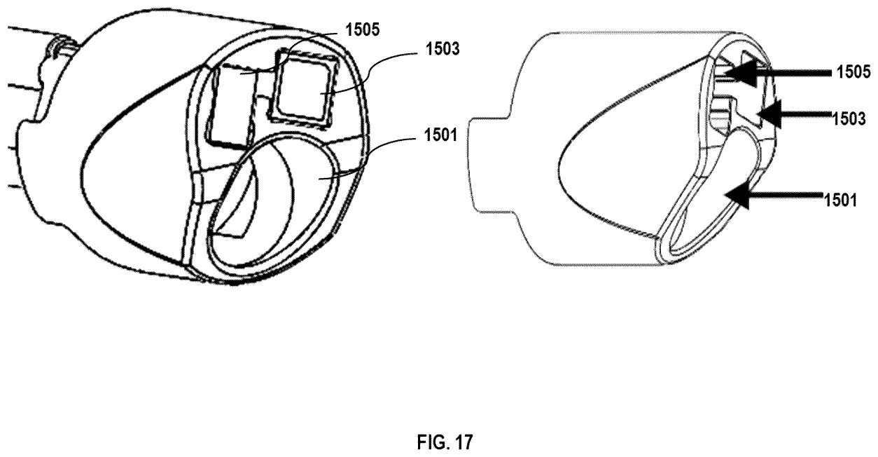

The angled tip may have one or more chamfered surfaces. In some cases, the chamfered portion of the tip may be substantially at the working channel 1501. As shown in FIG. 17, an exit port of the working channel 1501 may be at least partially chamfered. In some cases, at least 20%, 30%, 40%, 50%, 60%, 70%, 80%, 90% of an exit port of the working channel has a chamfered end surface. In some cases, the compartment for holding the imaging device/camera 1503 and the lighting/illumination device 1505 may be located substantially at the non-angled or non-chamfered surface such that the chambers may not impact the function of the camera or the lighting element.

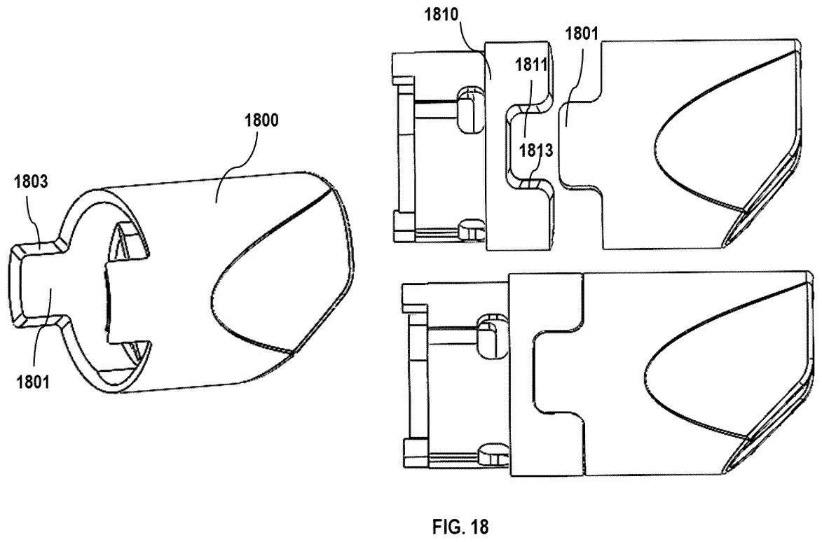

In some embodiments, the angled tip may comprise mating features to assist in aligning the angled tip to a connected shaft portion. As shown in FIG. 18, the back end of the tip portion 1800 may comprise one or more tab features 1801 to connect tip portion to the rest of the shaft 1810. The one or more tab features may provide both rotational alignment and lateral alignment. For example, the tab features 1801 once is coupled to the complementary mating feature 1811 formed on the shaft portion, the tab feature may prevent the tip from rotating relative to the shaft. In some cases, the tab feature may comprise a tapered surface 1803 that mates to the complementary feature 1813 of the shaft portion such that once the tip 1800 is assembled with the adjacent component 1810, the tab features lock the parts in place. The tapered sides of each of the tab features (and the corresponding mating features) may assist in lateral alignment. In some cases, once is tip is mated to the connected component, the tip portion may be adhered or welded in place.

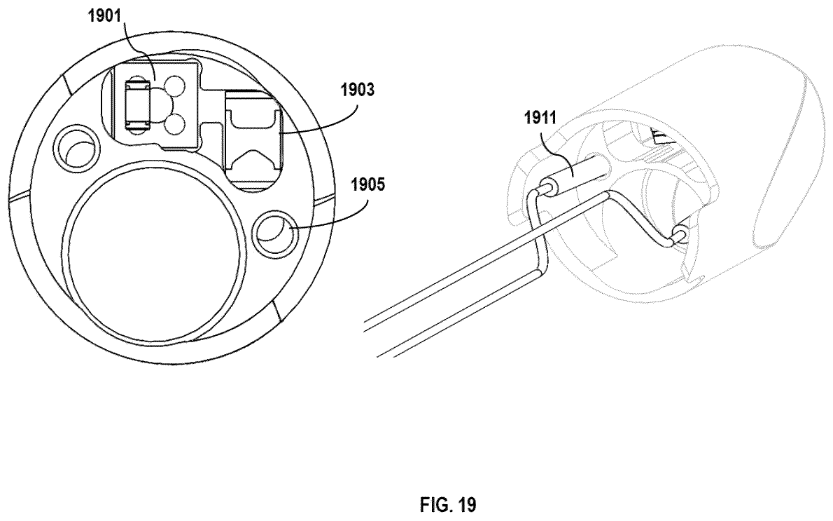

As described above, the angled or chamfered surfaces may reduce a diameter of the leading tip of the distal portion while allowing for one or more elements embedded at the tip. The one or more elements may comprise, for example, imaging device/camera, illumination device and/or position sensors (e.g., EM sensors). FIG. 19 shows an example of the angled tip with an embedded camera, an illumination device and one or more position sensors (e.g., EM sensors). As shown in the backend view of the tip portion, the rigid tip portion may have an internal structure such as a cutout to contain the camera 1901, and the lighting element 1903. The distal tip portion may further comprise structures 1905 to contain two EM sensors 1911.

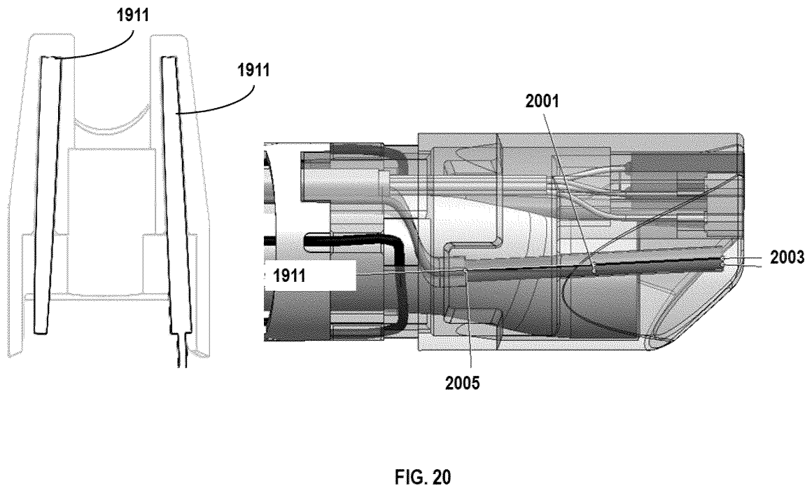

As further illustrated in FIG. 20, in some embodiments, the distal portion may contain a pair of EM sensors 1911 arranged in an angle. The angled arrangement (instead of parallel arrangement) beneficially allows for sensing information on the tip location and orientation with 6 degrees of freedom (i.e., parallel arrangement does not provide roll angle). The two EM sensors may be arranged at an angle 2001 with respect to the axial axis such that the two EM sensors are not parallel with respect to each other. In some cases, the two EM sensors may also be arranged at an angle 2003 with respect to a horizontal plane. In some cases, the EM sensors may be substantially located within the rigid tip portion. The rigid distal tip may have two angled holes 1905 that contain the EM sensors for navigation. The proximal end 2005 of the EM sensor may or may not be within the holes. In some cases, a portion of the EM sensor may be located within the hole 1905 while a proximal portion of the EM sensor may be extended to the connected shaft portion.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An articulatable flexible endoscope comprising:
an elongated member extending between a distal end and a proximal end, wherein the elongated member comprises a bending section steerable by one or more pull wires,
wherein the bending section comprises at least an inner flexure placed inside an outer flexure and wherein the inner flexure is formed with structures to provide structural support for the one or more pull wires upon assembly with the outer flexure; and
a distal tip portion connected to the distal end of the elongate member.

2. The articulating flexible endoscope of claim 1, wherein the inner flexure comprises repeated cut pattern at a first predetermined longitudinal pitch.

3. The articulating flexible endoscope of claim 2, wherein the outer flexure comprises repeated cut pattern at a second predetermined longitudinal pitch.

4. The articulating flexible endoscope of claim 3, wherein the second predetermined longitudinal pitch is a multiplication of the first predetermined longitudinal pitch.

5. The articulating flexible endoscope of claim 1, wherein the structures of the inner flexure comprise circumferential grooves and protrusions to form one or more lumens upon assembly with the outer flexure.

6. The articulating flexible endoscope of claim 5, wherein the distal tip portion comprises a structure to receive an imaging device, a position sensor, and an illumination device.

7. The articulating flexible endoscope of claim 6, wherein the one or more lumens provide structural support to one or more cables connected to the imaging device, the position sensor or the illumination device.

8. The articulating flexible endoscope of claim 6, wherein the distal tip portion has one or more chamfered surfaces.

9. The articulating flexible endoscope of claim 8, wherein the one or more chamfered surfaces are located substantially at a working channel.

10. The articulating flexible endoscope of claim 8, wherein the distal tip portion has a feature mating with a complementary feature of the bending section thereby providing a rotational alignment and lateral alignment.

11. The articulating flexible endoscope of claim 6, wherein the position sensor comprises a pair of electromagnetic sensors arranged at an angle.

12. The articulating flexible endoscope of claim 1, wherein the inner flexure is formed of a plurality of modular components connected via interlocking features.

13. The articulating flexible endoscope of claim 1, wherein the proximal end is connected to a proximal portion comprising a driving mechanism for applying a force to the one or more pull wires.

14. The articulating flexible endoscope of claim 1, wherein the proximal portion is supported by a robotic arm.

15. An articulating flexible endoscope comprising:
an elongated member extending between a distal end and a proximal end, wherein the elongated member comprises a bending section steerable by one or more pull wires,
wherein the bending section comprises a plurality of backbone components integrated into a plurality of polymer segments at various locations along the elongated member to vary a mechanical property of the bending section; and
a distal tip portion connected to the distal end of the elongate member.

16. The articulating flexible endoscope of claim 15, wherein the plurality of backbone components are thin disks formed of a material stiffer than a polymeric material.

17. The articulating flexible endoscope of claim 15, wherein the plurality of polymer segments have different lengths.

18. The articulating flexible endoscope of claim 17, wherein the different lengths are selected to adjust a minimum bending radius of the bending section.

19. The articulating flexible endoscope of claim 15, wherein the plurality of polymer segments have different configuration of lumens formed in each of the plurality of polymer segments.

20. The articulating flexible endoscope of claim 15, wherein at least one of the plurality of backbone components is fused into at least one of the plurality of polymer segments.
